(19) United States
(12) Patent Application Publication
(10) Pub. No.: US 2006/0112496 A1
(43) Pub. Date: Jun. 1, 2006

(54) Method for controlling spin-drying process with diagonal unbalance considered in spin-dryer or washing machine, and spin-dryer or washing machine controlling spin-drying process with the same

(76) Inventor: Gon Kim, Jiahae-si (KR)

Correspondence Address: FLESHNER & KIM, LLP, P.O. BOX 221200, CHANTILLY, VA, 20153, US

(21) Appl. No.: 11/289,417

(22) Filed: Nov. 30, 2005

(30) Foreign Application Priority Data
Dec. 1, 2004 (KR) 10-2004-99673

(51) Int. Cl. D06F 33/00; D06F 35/00; A01H 5/02

(52) U.S. Cl. 8/147; 8/158; 68/12.06; 68/23.1

(57) ABSTRACT

The present invention relates to a method for controlling a spin-drying process in a washing machine, and more particularly, to a method for controlling a spin-drying process in a washing machine with a diagonal unbalance considered, in which the spin-drying is performed considering the diagonal unbalance occurred if laundry is distributed in a direction, for an example, from an upper side of a front of a drum to a lower side of a rear of the drum, in a spin-drying course. The method for controlling a spin-drying course with a diagonal unbalance considered in a washing machine, includes a UB detection step for detecting a UB with a drum of the washing machine rotating, a UB comparison step for comparing said detected UB with a preset allowable UB, a diagonal UB detection step for detecting a diagonal UB and deciding whether a diagonal unbalance is allowable, if said detected UB is below said preset allowable UB in said UB comparison step, and a main spin-drying course step for entering a main spin-drying course so as to spin-dry wet clothes, if the diagonal unbalance is detected to be allowable.

### CROSS-REFERENCE TO RELATED APPLICATION

This application claims the benefit of Korean Application No. 10-2004-99673 filed on Dec. 1, 2004, which is hereby incorporated by reference as if fully set forth herein.

### BACKGROUND OF THE INVENTION

1. Field of the Invention

The present invention relates to a method for controlling a spin-drying process in a washing machine or a spin-dryer, and more particularly, to a method for controlling a spin-drying process in a washing machine or a spin-dryer with diagonal unbalance considered, in which the spin-drying is performed considering the diagonal unbalance occurred when laundry is distributed in the drum diagonally—for an example, some on an upper side of a front of a drum and some on a lower side of a rear of the drum—in a spin-drying course, and a washing machine or a spin-dryer controlling a spin-drying process with the same.

2. Discussion of the Related Art

In general, the washing machine removes dirt from laundry in a drum by using action of detergent and water in a tub, and the spin-dryer extracts water from wet laundry. Many related art washing machines also have a spin-drying function.

FIG. 1 illustrates a longitudinal section of a related art washing machine, FIG. 2 illustrates a front section of the drum in FIG. 1 at the time of occurrence of a diagonal unbalance schematically, and FIG. 3 illustrates a side section of the drum in FIG. 2.

Referring to FIG. 1, the related art washing machine is provided with a casing 2, a tub 10 in the casing 2 for holding washing water in washing or rinsing, and water extracted from laundry in a spin-drying course, a drum 20 rotatably mounted in the tub 10 for holding laundry ‘m’, and a motor 30 for supporting, and rotating the drum 20.

The casing 2 has a laundry opening 2a in one side for introduction of the laundry ‘m’, with a door 4 mounted thereon for opening/closing the laundry opening 2a.

The casing 2 is suspended with springs 5 over the casing 2 and dampers 6 under the casing 2.

The tub 10 has an opening 11 in rear of the laundry opening 2a.

Connected to the tub 10, there are a water supply unit 12 for supplying washing water to the tub 10 from an outside of the washing machine, and a draining unit 14 for draining the washing water from the tub 10 to the outside of the washing machine.

The drum 20 has an opening 21 for laundry ‘m’, washing water, and air, and holes 22 for washing water and air.

The motor 30 has a rotating shaft 32 passed through the tub 10 and supported on bearings mounted in the tub 10, with a fore end connected to the drum 20.

The motor 30 has sensing devices, such as RPM, or a hole sensor 34 for measuring a rotation angle.

Moreover, the washing machine is provided with a controller for controlling the water supply unit 12, the draining unit 14, and the motor 30 according to operation of the user, or sensed laundry, and unbalance amounts.

The operation of the related art washing machine will be described.

Upon putting the washing machine into operation after introduction of the laundry into the drum 20, and closing the door 4, the controller senses a laundry amount introduced thus, and sets a washing water level, a washing time period, rinsing and washing water flows, and so on.

The controller controls the water supply unit 12 for a set time period according to the laundry amount sensed thus, to supply the washing water to a preset water level of the washing machine, and the washing water supplied thus is held in the tub 10. Then, the controller drives the motor 30 at a preset time period at a preset speed, to rotate the drum 20, and the laundry ‘m’ in the drum 20 has dirt thereon removed by action of washing water. After finish of such a washing course, contaminated washing water is drained from the tub 10 to an outside of the washing machine through the draining unit 14.

In the meantime, in order to rinse foam form the laundry ‘m’, rinsing cycle is made for a few times, when, alike the washing cycle, the controller controls the water supply unit 12 and the motor 30 according to the sensed laundry amount. The contaminated water containing the foam is drained to the outside of the washing machine through the draining unit 14.

After the few times of rinsing cycle, the washing machine performs a spin-drying course for extracting water from the laundry ‘m’, centrifugally.

The spin-drying course is provided with laundry sensing for sensing a laundry amount for determining an optimum spin-drying time period or spin-drying RPM, UB detection for detecting an unbalance amount (UB) before starting a main spin-drying course, and a main spin-drying for running the motor 30 at a high speed after the detection of the laundry amount or the unbalance amount.

The term ‘UB’ is used to indicate the degree of unbalance of the drum with wet clothes. The unbalance causes undesirable vibration and noise to take place in the machine when the drum rotating. The UB can be detected in many ways. For example, the UB can be detected by the equation as below.
UB=k×I×RPMᵣᵢₚₚₗₑ

(k is a constant obtained by an experiment, I is the moment of inertia of wet clothes in the drum, and RPMᵣᵢₚₚₗₑ is the magnitude of a ripple of the rpm of the motor rotating the drum when the motor is controlled to be rotated in a constant speed.)

The laundry amount is detected by turning off the motor 30 after running the motor 30 at a constant speed for a predetermined time period, and measuring a pulse width modulation (PWM) duty value from starting of the motor 30 to turning off of the motor 30, and an inertial rotation angle of the motor 30 after the turning off of the motor 30.

Then, the washing machine rotates the drum 20 in one direction to detect a UB.

If the UB detected thus is lower than a preset allowable UB, the washing machine determines that the laundry is not unbalanced, and rotates the drum 20 in the same direction at a high speed, to perform a main spin-drying course.

Opposite to this, if the UB detected thus is higher than the preset allowable UB, the washing machine determines that the laundry is unbalanced, performs relocation of laundry in which the drum 20 is rotated in regular/reverse directions alternately until the UB is lower than the preset allowable UB, and performs the main spin-drying course.

The washing machine performs the main spin-drying course for the spin-drying time period at a spin-drying RPM set at the laundry detection, to spin-dry the laundry at a high speed, the draining unit 14 drains water from the laundry to the outside of the washing machine, and the washing machine stops the drum 20 when the spin-drying time period is passed, to terminate the spin-drying course of the washing machine.

However, referring to FIGS. 2 and 3, the related art method for controlling a spin-drying process has a problem in that detection of a diagonal unbalance is failed. The inventor verified that such a diagonal unbalance can not be detected at the drum rotation speed in the related art unbalance detection. Consequently, in the related art, the UB is detected to be below the preset allowable UB even if there is a diagonal unbalance, to determine that the UB is an allowable level to permit the washing machine to start the main spin-drying course, resulting in heavy vibration and noise.

### SUMMARY OF THE INVENTION

Accordingly, the present invention is directed to a method for controlling a spin-drying process in a washing machine or a spin-dryer with a diagonal unbalance considered, and a washing machine or a spin-dryer controlling a spin-drying process with the same that substantially obviates one or more problems due to limitations and disadvantages of the related art.

An object of the present invention is to provide a method for controlling a spin-drying process with a diagonal unbalance considered, in which a diagonal unbalance is detected, to start a main spin-drying course only when the diagonal unbalance is within an allowable level, for preventing vibration and noise caused by the diagonal unbalance.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as appended drawings.

To achieve these objects and other advantages and in accordance with the purpose of the invention, as embodied and broadly described herein, a method for controlling a spin-drying course with a diagonal unbalance considered in a washing machine includes a UB detection step for detecting a UB with a drum of the washing machine rotating; a UB comparison step for comparing the detected UB with a preset allowable UB; a diagonal UB detection step for detecting a diagonal UB and deciding whether a diagonal unbalance is allowable, if the detected UB is below the preset allowable UB in the UB comparison step; and a main spin-drying course step for entering a main spin-drying course so as to spin-dry wet clothes, if the diagonal unbalance is detected to be allowable.

The diagonal UB detection step can comprise: a UB redetection step for redetecting a UB with the drum rotating in a higher speed than a speed in the UB detection step; a redetected UB comparison step for comparing the redetected UB with a preset allowable diagonal UB; a diagonal unbalance decision step for deciding that the diagonal unbalance is not allowable if the redetected UB is the same as or over an allowable diagonal UB.

The preset allowable UB is a UB already set to be allowable and the preset allowable diagonal UB is also a UB already set to be allowable with a diagonal unbalance considered. The preset allowable UB and the preset allowable diagonal UB can be obtained by experiments.

The present invention can further comprise a clothes relocation step for relocating wet clothes in the drum so as to decrease the diagonal unbalance, if the diagonal unbalance is detected not to be allowable. The clothes relocation step can be accomplished by repeatedly rotating the drum in a direction and the other in turn such that the wet clothes in the drum can be dispersed.

The diagonal UB detection step and the clothes relocation step can be repeated until the diagonal unbalance is detected to be allowable in the diagonal UB detection step.

Another form of the present invention is a method for controlling a spin-drying course with a diagonal unbalance considered in a washing machine, wherein the method comprises: a diagonal UB detection speed rotation step for rotating a drum of the washing machine in a rotational speed of a diagonal UB detection speed; a UB detection step for detecting a UB with the drum rotating in the rotational speed of the diagonal UB detection speed; a UB comparison step for comparing the detected UB with a preset allowable UB; a diagonal unbalance considered control step for controlling a spin-drying course of the washing machine according to the comparison result of the UB comparison step.

The diagonal UB detection speed is a rotational speed of the drum in which a diagonal UB can be detected. A diagonal unbalance could not be detected with a conventional method for detecting an unbalance which is general, and the machine was controlled to enter a main spin-drying course even if a diagonal unbalance existed. The inventor found that the rotational speed of the drum in detecting unbalance was too low to detect the diagonal unbalance and the diagonal unbalance did not cause vibration or noise in that low speed enough to be noticed or detected.

Therefore, in a conventional machine, a main spin-drying course where the drum rotates in even higher speed such as 1200 rpm was entered if a UB was detected to be lower than a preset allowable UB even though a diagonal unbalance existed. The diagonal unbalance caused heavy vibration and noise in the high rotational speed of the main spin-drying course.

The inventor had various tries to solve the problem of a diagonal unbalance not being detected, and in the end he found that the rotational speed must be higher to well detect a diagonal unbalance. For example, a diagonal unbalance was detected in the rotational speed of about 350 rpm of the drum. Conventionally, the drum was rotated in the speed of about 170 rpm in detecting a UB which is general.

The invention can further comprise: a step for deciding to go to the diagonal UB detection speed rotation step, if a UB is below the preset allowable UB after being detected with the drum rotating in a lower rotational speed than the diagonal UB detection speed.

The preset allowable UB can be a preset allowable diagonal UB.

The diagonal unbalance considered control step can comprise a clothes relocation step for relocating wet clothes in the drum so as to decrease the diagonal unbalance, if the detected UB is the same as or over the preset allowable UB.

The detection of the UB in the UB detection step can be accomplished by using a rpm ripple of a motor rotating the drum in the speed of the diagonal UB detection speed. For example, the above-mentioned equation can be used.

Another form of the present invention is a washing machine performing a spin-drying course with a diagonal unbalance considered, wherein the washing machine comprises: a diagonal UB detection speed rotation means for rotating a drum of the washing machine in a rotational speed of a diagonal UB detection speed; a UB detection means for detecting a UB with the drum rotating in the rotational speed of the diagonal UB detection speed; a UB comparison means for comparing the detected UB with a preset allowable UB; a diagonal unbalance considered control means for controlling a spin-drying course of the washing machine according to the comparison result from the UB comparison means.

The invention can further comprise a means for detecting a UB with the drum rotating in a lower rotational speed than the diagonal UB detection speed and deciding to rotate the drum in the speed of the diagonal UB detection speed if the detected UB is below the preset allowable UB.

The preset allowable UB can be a preset allowable diagonal UB.

The diagonal unbalance considered control means can comprise a clothes relocation means for relocating wet clothes in the drum so as to decrease the diagonal unbalance, if the detected UB is the same as or over the preset allowable UB.

The detection of the UB in the UB detection means can be accomplished by using a rpm ripple of a motor rotating the drum in the speed of the diagonal UB detection speed.

Another form of the present invention is a method for controlling a spin-drying course with a diagonal unbalance considered in a spin-drying machine, wherein the method comprises: a diagonal UB detection speed rotation step for rotating a drum of the spin-drying machine in a rotational speed of a diagonal UB detection speed; a UB detection step for detecting a UB with the drum rotating in the rotational speed of the diagonal UB detection speed; a UB comparison step for comparing the detected UB with a preset allowable UB; a diagonal unbalance considered control step for controlling a spin-drying course of the spin-drying machine according to the comparison result of the UB comparison step.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings;

FIG. 1 illustrates a longitudinal section of a related art washing machine;

FIG. 2 illustrates a front section of the drum in FIG. 1 at the time of occurrence of a diagonal unbalance schematically;

FIG. 3 illustrates a side section of the drum in FIG. 2;

FIG. 4 illustrates a flow chart showing the steps of a method for controlling a spin-drying process with a diagonal unbalance considered in a washing machine in accordance with a preferred embodiment of the present invention; and

FIG. 5 illustrates a graph of a spin-drying time period vs. a motor RPM in a washing machine in accordance with a preferred embodiment of the present invention.

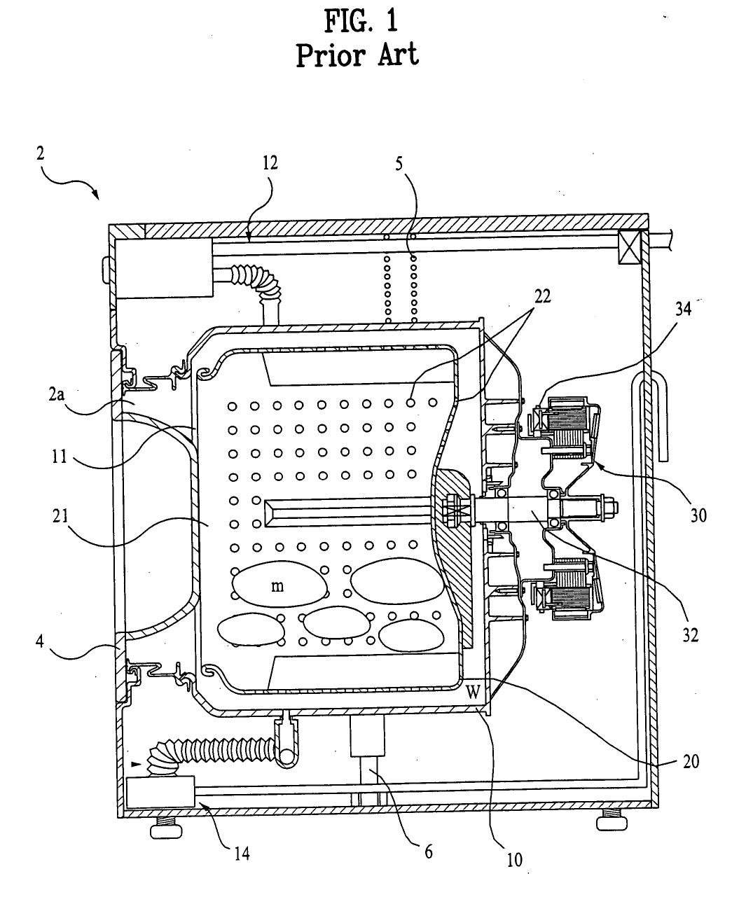

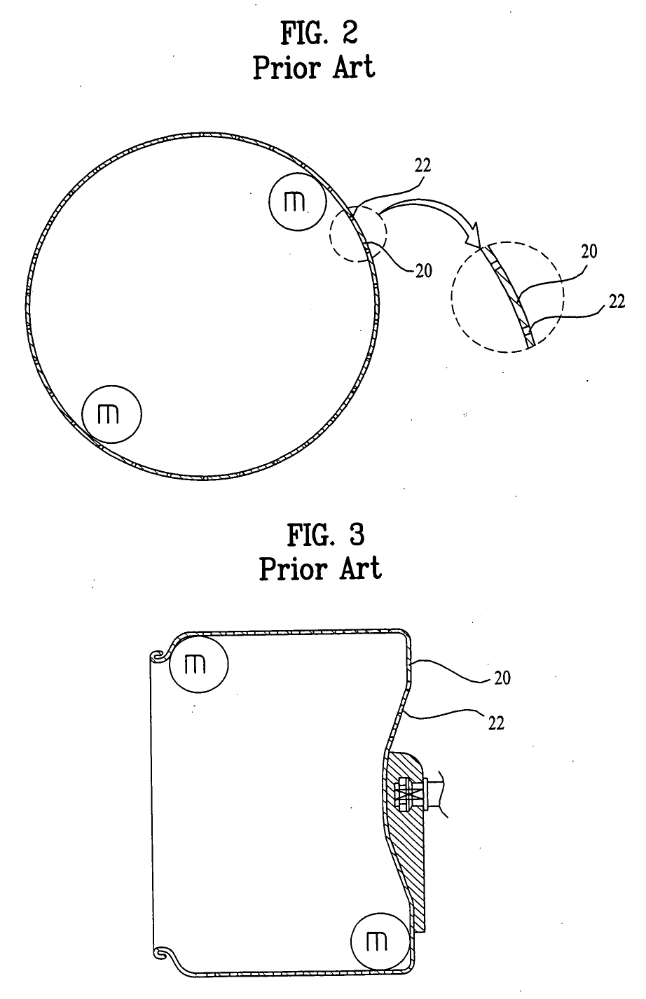

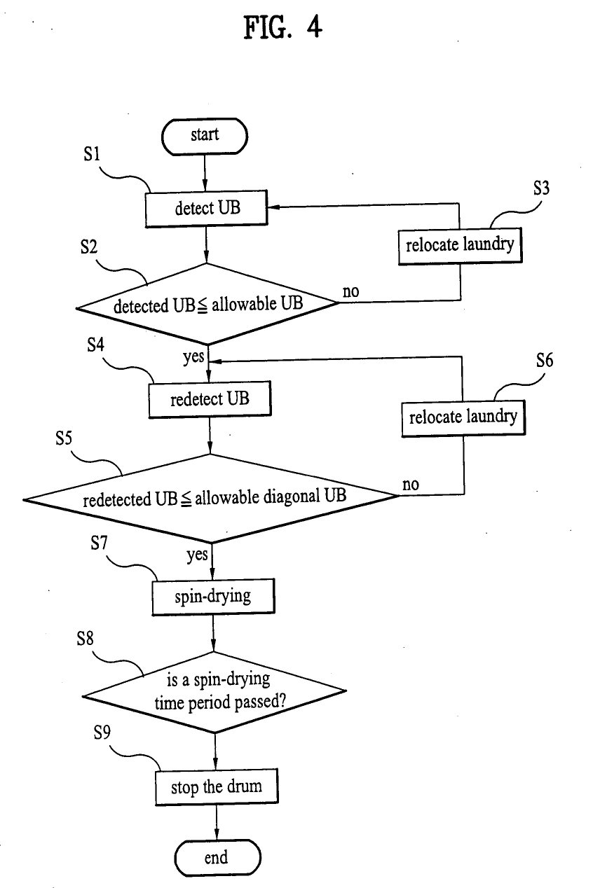

### DETAILED DESCRIPTION OF THE INVENTION

Reference will now be made in detail to the preferred embodiments of the present invention, examples of which are illustrated in the accompanying drawings. Wherever possible, the same reference numbers will be used throughout the drawings to refer to the same or like parts.

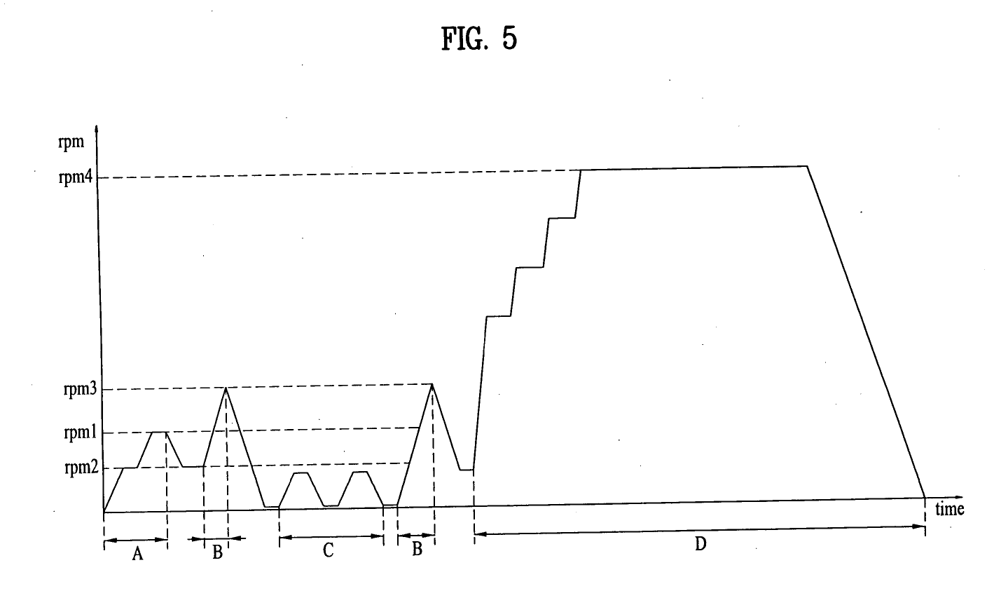

FIG. 4 illustrates a flow chart showing the steps of a method for controlling a spin-drying process with a diagonal unbalance considered in a washing machine in accordance with a preferred embodiment of the present invention, and FIG. 5 illustrates a graph of a spin-drying time period vs. a motor RPM in a washing machine in accordance with a preferred embodiment of the present invention.

Referring to FIGS. 4 and 5, in the method for controlling a spin-drying process with a diagonal unbalance considered in a washing machine, a drum 20 is rotated, to detect an unbalance amount (UB) (S1, A section).

Referring to FIG. 5, the UB is detected by accelerating the motor 30 to a first set RPM rpm_1, and detecting a rpm ripple at the first set RPM rpm_1 which is taken as the UBcw1, or other methods.

Then, referring to FIG. 4, the UB detected thus is compared to a preset allowable UB (S2).

The preset allowable UB is a preset allowable UB for determining whether the unbalance is allowable for the drum 20 to be rotated in a high speed (i.e., a main spin-drying) or not.

As result of the comparison, if the UB detected is greater than the preset allowable UB, determining that the laundry is unbalanced in the drum 20, relocation of the laundry is performed for resolving the unbalance (S3).

In the relocation of the laundry, the motor 30 is rotated in regular/reverse directions at a second set RPM rpm_2 (rpm_2<rpm_{—1}) so that the drum 20 rotates in a clockwise direction and a counter clockwise direction, alternately.

After relocation of the laundry, the washing machine detects the UB again, to repeat the detection of the UB, the comparison to the preset allowable UB, and the relocation of the laundry until the UB detected is smaller than the preset allowable UB.

Opposite to this, as a result of the comparison, if the UB detected is smaller than the preset allowable UB, determining that the laundry is not unbalanced in the drum 20, a diagonal unbalance detection is performed.

The predetermined time period is a preset standby time period for enhancing an accuracy of the diagonal unbalance detection.

In the diagonal unbalance detection, if the predetermined time period is passed after a speed of the drum 20 is reduced, the motor 30 is controlled such that the drum 20 can be rotated at a speed higher than the speed of the drum 20 at the time of the unbalance detection, and the UB is redetected while the drum 20 rotates at the high speed (S4, B section).

The washing machine accelerates the motor 30 until the motor 30 reaches to a third set RPM rpm_3 (rpm_3>rpm_{—}1) higher than the first set RPM rpm_1, when the washing machine takes a rpm ripple to detect the UB, or the washing machine detects the UB by other method.

Then, the washing machine compares the UB redetected thus to the preset allowable diagonal UB, to determine that there is a diagonal unbalance if the redetected UB is greater than the preset allowable diagonal UB, or to determine that a diagonal unbalance, if any, is allowable if the redetected UB is smaller than the preset allowable diagonal UB (S5).

The preset allowable diagonal UB is a preset reference unbalance amount for determining whether the drum 20 is unbalanced diagonally or not, and may be set equal to, or greater than the preset allowable UB.

In the case of the laundry in the drum 20 being unbalanced diagonally, if the UB is redetected while the drum 20 is rotated at a speed higher than the speed when the UB is detected after a predetermined time period is passed from reducing the speed of the drum 20, the UB redetected thus will be greater than the UB at the time of the detection of unbalance (S1). Therefore, if the redetected UB is greater than the preset allowable diagonal UB, it means there is a diagonal unbalance not allowable at the drum 20, and if the redetected UB is smaller than the preset allowable diagonal UB, it means a diagonal unbalance, if any, is allowable at the drum 20.

If it is determined that the washing machine has the diagonal unbalance not allowable, a laundry relocation is performed for decreasing the diagonal unbalance at the drum 20 (S6, C section).

After the relocation of the laundry, the washing machine controls the motor 30 so that the drum 20 is rotated at a high speed after a predetermined time passed from reducing the speed of the drum 20, the UB is redetected while the drum 20 is rotated at the high speed, and repeats the redetection of the UB, the comparison to the preset allowable diagonal UB, and the relocation of the laundry until the UB redetected is smaller than the preset allowable diagonal UB (S4, S5, and S6).

On the other hand, if it is determined that the washing machine is not unbalanced diagonally, the drum speed is reduced, and the spin-drying is started after a predetermined time period is passed (S7, D section).

The washing machine accelerates the motor 30 in multiple steps until RPM of the motor 30 reaches to a fourth set RPM rpm_4 (rpm_4>rpm_3) higher than the third set RPM rpm_3, when the washing machine rotates the motor 30 at the fourth set RPM rpm_4 for the spin-drying time period (S7).

Of course, the spin-drying time period and the fourth set RPM may be set according to the laundry amount detection performed before the initial UB detection, or may be set regardless of the laundry amount.

While the motor 30 is rotated at the fourth set RPM for the spin-drying time period, the laundry in the drum 20 is spin-dried by the high speed rotation of the drum centrifugally, and if the fourth spin-drying time period is passed, the washing machine turns off the motor 30 to stop the drum 20.

As has been described, the method for controlling a spin-drying process with a diagonal unbalance considered in a washing machine has the following advantages.

Even if it is determined that laundry in a drum is not unbalanced, a spin-drying course is not started directly, but a diagonal unbalance of the laundry is determined additionally, to start the spin-drying if it is determined that the laundry is not unbalanced diagonally, thereby permitting to prevent vibration and noise caused by the diagonal unbalance.

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents.

What is claimed is:

1. A method for controlling a spin-drying course with a diagonal unbalance considered in a washing machine, wherein the method comprises:
a UB detection step for detecting a UB with a drum of the washing machine rotating;
a UB comparison step for comparing said detected UB with a preset allowable UB;
a diagonal UB detection step for detecting a diagonal UB and deciding whether a diagonal unbalance is allowable, if said detected UB is below said preset allowable UB in said UB comparison step; and
a main spin-drying course step for entering a main spin-drying course so as to spin-dry wet clothes, if the diagonal unbalance is detected to be allowable.

2. The method of claim 1, wherein said diagonal UB detection step comprises:
a UB redetection step for redetecting a UB with the drum rotating in a higher speed than a speed in said UB detection step;
a redetected UB comparison step for comparing said redetected UB with a preset allowable diagonal UB;
a diagonal unbalance decision step for deciding that the diagonal unbalance is not allowable if said redetected UB is the same as or over an allowable diagonal UB.

3. The method of claim 1, wherein the method further comprises:
a clothes relocation step for relocating wet clothes in the drum so as to decrease the diagonal unbalance, if the diagonal unbalance is detected not to be allowable.

4. The method of claim 2, wherein the method further comprises:
a clothes relocation step for relocating wet clothes in the drum so as to decrease the diagonal unbalance, if the diagonal unbalance is detected not to be allowable.

5. The method of claim 3, wherein said diagonal UB detection step and said clothes relocation step are repeated until the diagonal unbalance is detected to be allowable.

6. The method of claim 4, wherein said diagonal UB detection step and said clothes relocation step are repeated until the diagonal unbalance is detected to be allowable.

7. A method for controlling a spin-drying course with a diagonal unbalance considered in a washing machine, wherein the method comprises:
a diagonal UB detection speed rotation step for rotating a drum of the washing machine in a rotational speed of a diagonal UB detection speed;
a UB detection step for detecting a UB with the drum rotating in the rotational speed of said diagonal UB detection speed;
a UB comparison step for comparing said detected UB with a preset allowable UB;
a diagonal unbalance considered control step for controlling a spin-drying course of the washing machine according to the comparison result of said UB comparison step.

8. The method of claim 7, wherein the method further comprises:
a step for deciding to go to said diagonal UB detection speed rotation step, if a UB is below said preset allowable UB after being detected with the drum rotating in a lower rotational speed than said diagonal UB detection speed.

9. The method of claim 7, wherein said preset allowable UB is a preset allowable diagonal UB.

10. The method of claim 7, wherein said diagonal unbalance considered control step comprises:
a clothes relocation step for relocating wet clothes in the drum so as to decrease the diagonal unbalance, if the detected UB is the same as or over the preset allowable UB.

11. The method of claim 7, wherein the detection of the UB in said UB detection step is accomplished by using a rpm ripple of a motor rotating the drum in the speed of said diagonal UB detection speed.

12. A washing machine performing a spin-drying course with a diagonal unbalance considered, wherein the washing machine comprises:
a diagonal UB detection speed rotation means for rotating a drum of the washing machine in a rotational speed of a diagonal UB detection speed;
a UB detection means for detecting a UB with the drum rotating in the rotational speed of said diagonal UB detection speed;
a UB comparison means for comparing said detected UB with a preset allowable UB;
a diagonal unbalance considered control means for controlling a spin-drying course of the washing machine according to the comparison result from said UB comparison means.

13. The washing machine of claim 12, wherein the washing machine further comprises:
a means for detecting a UB with the drum rotating in a lower rotational speed than said diagonal UB detection speed and deciding to rotate the drum in the speed of said diagonal UB detection speed if the detected UB is below said preset allowable UB.

14. The washing machine of claim 12, wherein said preset allowable UB is a preset allowable diagonal UB.

15. The washing machine of claim 12, wherein said diagonal unbalance considered control means comprises:
a clothes relocation means for relocating wet clothes in the drum so as to decrease the diagonal unbalance, if the detected UB is the same as or over the preset allowable UB.

16. The washing machine of claim 12, wherein the detection of the UB in said UB detection means is accomplished by using a rpm ripple of a motor rotating the drum in the speed of said diagonal UB detection speed.

17. A method for controlling a spin-drying course with a diagonal unbalance considered in a spin-drying machine, wherein the method comprises:
a diagonal UB detection speed rotation step for rotating a drum of the spin-drying machine in a rotational speed of a diagonal UB detection speed;
a UB detection step for detecting a UB with the drum rotating in the rotational speed of said diagonal UB detection speed;
a UB comparison step for comparing said detected UB with a preset allowable UB;
a diagonal unbalance considered control step for controlling a spin-drying course of the spin-drying machine according to the comparison result of said UB comparison step.
